(19) United States
(12) Patent Application Publication
(10) Pub. No.: US 2025/0281271 A1
(43) Pub. Date: Sep. 11, 2025

(54) TISSUE STORAGE, IRRIGATION, AND INFUSION MEDIUM AND METHODS OF USE

(71) Applicant: NEW YORK SOCIETY FOR THE RELIEF OF THE RUPTURED AND CRIPPLED, MAINTAINING THE HOSPITAL FOR SPECIAL, New York, NY (US)

(72) Inventors: Guang-Ting Cong, Lloyd Harbor, NY (US); Sheeraz Qureshi, New York, NY (US); Matthew E. Cunningham, Lloyd Harbor, NY (US); Kyle Morse, New York, NY (US)

(21) Appl. No.: 18/858,176

(22) PCT Filed: Apr. 20, 2023
(86) PCT No.: PCT/US2023/019273

§ 371 Date: Oct. 18, 2024

Related U.S. Application Data
(60) Provisional application No. 63/333,369, filed on Apr. 21, 2022.
(60) Provisional application No. 63/415,506, filed on Oct. 12, 2022.

(51) Int. Cl. A61D 7/00; A01N 1/126; A61K 9/00; A61K 31/546; A61K 31/7036; A61K 31/7048; A61K 35/16; A61K 38/14; A61K 47/02; A61K 47/18; A61K 47/22; A61K 47/26

(52) U.S. Cl. CPC A61D 7/00; A01N 1/126; A61K 9/0017; A61K 9/0019; A61K 31/546; A61K 31/7036; A61K 31/7048; A61K 35/16; A61K 38/14; A61K 47/02; A61K 47/183; A61K 47/22; A61K 47/26

(57) ABSTRACT

This disclosure features tissue storage, irrigation, and infusion media compositions, and methods of using such compositions, for example, the temporary storage of cells and tissues prior to implantation or re-implantation, or for wound irrigation, wound packing, surgical site irrigation, or intravenous infusion. The tissue storage, irrigation, and infusion media compositions disclosed herein are useful in a healthcare or veterinary setting.

### CROSS-REFERENCE TO RELATED APPLICATIONS

This application claims the benefit of U.S. Provisional Patent Application No. 63/333,369, filed Apr. 21, 2022, and U.S. Provisional Patent Application No. 63/415,506, filed Oct. 12, 2022, the contents of which are incorporated herein by reference in their entirety.

### TECHNICAL FIELD

This disclosure relates to tissue storage, irrigation, and infusion medium compositions and methods for the temporary storage of cells and tissues and the irrigation of wounds and sites of surgical procedures, or intravenous infusion, in a healthcare or veterinary setting.

### BACKGROUND

In healthcare or veterinary medicine, the harvesting and re-implantation of cells and tissue is a common method used to supplement healing. However, cells and tissue are prone to changes in environmental and biological conditions prior to, during, and following re-implantation, which may impact tissue survival and negatively impact clinical outcomes. Many environmental and biological conditions negatively impact cell and tissue survival when they are stored outside of the body. Examples include changes in temperature, changes in pH, tissue ischemia, mechanical handling, amongst others. For instance, the most immediate and critical problem is cell and tissue exposure to air drying effects and loss of osmotic balance. Direct exposure to air drying can kill cells within seconds, and deprivation of critical nutrients lead to cell death within minutes. Thus, there is a need for safe and effective temporary storage of live cells and tissue prior to re-implantation.

Further, wound irrigation can be an important part of wound management and use of wound irrigation in wound care may reduce the risk of infection during wound closure and wound management. The goal of wound irrigation is to remove foreign material from the wound, decrease bacterial contamination of the wound, and to remove cellular debris or exudate from the surface of the wound, without negatively affecting the host tissue.

Still further, during surgical procedures, surgical site infections are costly and are associated with poorer patient outcomes. Accordingly, intra-operative surgical site irrigation can reduce the risk of surgical site infections by removing dead or damaged tissue, bacterial contaminants, metabolic waste, and surgical site exudate. Irrigation with antibiotic or antiseptic solutions may further reduce the risk of surgical site infections during procedures.

Additionally, after intravenous infusion, for example as a maintenance or resuscitation intravenous fluid for use in healthcare, field medicine, or veterinary care, even short-time exposure of venous or arterial intimal endothelium to physiologic salines or crystalloids creates intimal tissue damage. Some currently available resuscitation media use a 1:1:1 ratio of blood:plasma:platelets which, while effective at reducing trauma mortality, is highly limited by the availability of donor human blood products and cost. Short of readily provisioned transfusible donor blood products, a transfusible tissue medium-derived intravenous fluid causes less cellular and tissue injury as compared to medical-grade salines, and may thus act as an artificial plasma, or at minimum a robust tissue-friendly saline replacement.

Contact with compositions used as infusion media or irrigation media, for example intravenous-grade saline, may compromise the viability of cells and tissues at the site of infusion or irrigation, even after exposure of less than one hour. Local tissue damage caused by crystalloids may therefore potentiate pain and cause increased infection rates, such as that demonstrated in the laparascopic surgery literature. Thus, there is a need for infusion media and irrigation media that promote the viability of cells and tissues at the site of infusion or irrigation, for example, at wounds, surgical sites, or vascular endothelial tissues.

### SUMMARY

This disclosure relates to tissue storage, irrigation, and infusion medium compositions and methods for the temporary storage of cells and tissues containing live cells prior to implantation or re-implantation, and for the irrigation of wounds and sites of surgical procedures, or intravenous infusion, in a healthcare or veterinary setting.

In a first aspect, the disclosure provides methods of irrigating a tissue of a subject, the methods including exposing the tissue to a liquid medium that does not contain serum or growth factors. In some embodiments, the liquid medium does not contain peptides. In some embodiments, the tissue is at a site of a wound in a subject. In some embodiments, the site of the wound has been treated with a wound packing and/or vacuum device. In some embodiments, the tissue is at a surgical site in a subject. In some embodiments, the surgical site is an arthroscopic surgical site, an endoscopic surgical site, a microscopic surgical site, or a dental surgical site. In some embodiments, the subject is a human or a non-human mammal. In some embodiments, the infusion and/or irrigation medium is Dulbecco's Modified Eagle Medium (DMEM) or alpha-minimum essential medium (aMEM) or similar compositions thereof. In some embodiments, the infusion and/or irrigation medium is a sterile cell culture medium. In some embodiments, the infusion and/or irrigation medium has a similar composition to human plasma or human plasma-like medium (HPLM). In some embodiments, the infusion and/or irrigation medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium. In some embodiments, the infusion and/or irrigation medium further comprises one or more antibiotic ingredients. In some embodiments, the antibiotic ingredient is cefazolin, vancomycin, tobramycin, gentamicin, or amphotericin. In some embodiments, the tissue comprises one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma. In some embodiments, the cells of the tissue contacted by the infusion and/or irrigation medium have greater viability compared to cells of a second tissue contacted by an intravenous-grade saline solution. In some embodiments, the healthcare or veterinarian setting is a sterile environment. In some embodiments, the healthcare setting is an organized provision of medical care to individuals, or to a community. In some embodiments, the veterinarian setting is an organized provision of medical care to animals. In some embodiments, the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

In another aspect, the disclosure provides methods of providing an intravenous infusion to a subject, including intravenously providing a composition including tissue culture medium that does not contain serum, or growth factors. In some embodiments, the composition does not contain peptides. In some embodiments, the composition further includes one or more antibiotic ingredients. In some embodiments, the antibiotic ingredient is cefazolin, vancomycin, tobramycin, gentamicin, or amphotericin.

In another aspect, the disclosure provides compositions including an infusion and/or irrigation medium, wherein the infusion and/or irrigation medium includes the following components: water, glucose or fructose, sodium, potassium, chloride, bicarbonate, and at least one amino acid; wherein the infusion and/or irrigation medium does not contain serum or growth factors. In some embodiments, the infusion and/or irrigation medium does not contain peptides. In some embodiments, the infusion and/or irrigation medium further includes a combination of one or more of the following components: lactate, galactose, acetate, gluconate, thiamine, riboflavin, niacin, pantothenic acid, para-aminobenzoic acid, pyridoxine, biotin, folates, cobalamin, inositol, vitamin A, vitamin D, vitamin E, vitamin K, vitamin C, L-alanine, L-arginine, L-asparagine, L-aspartic acid, L-cysteine, L-cystine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, taurine, glutathione, glucuronolactone, acetone, carnitine, creatine, creatinine, glycerol, hypoxanthine, sodium pyruvate, urea, uridine, acetylcarnitine, betaine, L-amino-N-butyric acid, L-citrulline, L-hydroxyproline, L-ornithine, N-acetylglycine, 3-hydroxybutyric acid, alpha ketoglutarate, citric acid, formic acid, malic acid, malonic acid, succinic acid, uric acid adenine, adenosine, deoxyadenosine, guanine, guanosine, deoxyguanosine, cytosine, cytidine, deoxycytidine, thymine, thymidine, deoxythymidine, uracil, dietary fatty acids (including saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), lipoic acid, cholesterol, bisphosphonates, ammonium, sulfate, chloride, phosphate, bicarbonate, nitrate, and/or inorganic ionic forms of elemental magnesium, calcium, boron, zinc, copper, iron, phosphorous, vanadium, strontium, silica, silicon, selenium, nickel, or manganese; wherein the composition has a near-iso-osmotic balance of 170 to 406 mOsm/L and a pH of 6.0 to 8.8 as a sterile aqueous solution. In some embodiments, the infusion and/or irrigation medium has a pH of 5.0 to 9.0. In some embodiments, the infusion and/or irrigation medium further comprises the following components: water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.1-10 mM), sodium, potassium, chloride, and bicarbonate. In some embodiments, the infusion and/or irrigation medium comprises the following components: water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has a near-iso-osmotic balance of 170 to 406 mOsm/L). In some embodiments, the composition further comprises one or more antibiotic ingredients. In some embodiments, the antibiotic ingredient is cefazolin, vancomycin, tobramycin, gentamicin, or amphotericin.

In another aspect, the disclosure provides methods of irrigating a tissue of a subject, the method including exposing the tissue to a liquid medium that does not contain serum or growth factors. In some embodiments, the tissue is at the site of a wound in a subject. In some embodiments, the site of the wound has been treated with a wound packing and/or vacuum device. In some embodiments, the tissue is at a surgical site in a subject. In some embodiments, the surgical site is an arthroscopic surgical site, an endoscopic surgical site, a microscopic surgical site, or a dental surgical site. In some embodiments, the subject is a human or a non-human mammal. In some embodiments, the infusion and/or irrigation medium is Dulbecco's Modified Eagle Medium (DMEM) or alpha-minimum essential medium (aMEM) or similar compositions thereof. In some embodiments, the infusion and/or irrigation medium is a sterile cell culture medium. In some embodiments, the infusion and/or irrigation medium has a similar composition to human plasma or human plasma-like medium (HPLM). In some embodiments, the infusion and/or irrigation medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium. In some embodiments, the infusion and/or irrigation medium further comprises one or more antibiotic ingredients. In some embodiments, the antibiotic ingredient is cefazolin, vancomycin, tobramycin, gentamicin, or amphotericin.

In some embodiments, the tissue includes one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma. In some embodiments, the cells of the tissue contacted by the infusion and/or irrigation medium have greater viability compared to cells of a second tissue contacted by an intravenous-grade saline solution. In some embodiments, the healthcare or veterinarian setting is a sterile environment. In some embodiments, the healthcare setting is an organized provision of medical care to individuals, or to a community. In some embodiments, the veterinarian setting is an organized provision of medical care to animals. In some embodiments, the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

In another aspect, the disclosure provides methods of providing an intravenous infusion to a subject, including intravenously providing a composition including tissue culture medium that does not contain serum or growth factors. In some embodiments, the composition further includes one or more antibiotic ingredients.

In another aspect, the disclosure provides methods of irrigating a tissue of a subject, the methods including exposing the tissue to a liquid medium that does not contain serum or growth factors. In some embodiments, the liquid medium does not contain peptides.

In another aspect, the disclosure provides methods of providing an intravenous infusion to a subject, including intravenously providing a composition including tissue culture medium that does not contain serum or growth factors. In some embodiments, the tissue culture medium does not contain peptides.

In another aspect, the disclosure provides methods of irrigating a tissue of a subject, the method including exposing the tissue to a liquid medium that does not contain peptides or serum.

In another aspect, the disclosure provides methods of providing an intravenous infusion to a subject, including intravenously providing a composition including tissue culture medium that does not contain peptides or serum.

In another aspect, the disclosure provides methods of storing tissue obtained from a subject, the method including exposing the tissue to a tissue culture medium that does not contain serum, or growth factors, and reserving the exposed tissue for re-implantation in the subject. In some embodiments, the tissue culture medium does not contain peptides. In some embodiments, the tissue is stored for less than about 96 hours after obtaining the tissue from the subject. In some embodiments, the subject is a human or a non-human mammal. In some embodiments, the tissue culture medium is Dulbecco's Modified Eagle Medium (DMEM) or alpha-minimum essential medium (aMEM) or similar compositions thereof. In some embodiments, the tissue culture medium is a sterile enriched cell culture medium. In some embodiments, the tissue culture medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium. In some embodiments, the tissue culture medium further comprises one or more antibiotic ingredients. In some embodiments, the antibiotic ingredient is cefazolin, vancomycin, tobramycin, gentamcin, or amphotericin. In some embodiments, the tissue is a non-whole-organ tissue. In some embodiments, the tissue is a stem cell-containing tissue or a cell fraction. In some embodiments, the stem cell-containing tissue is bone marrow aspirate or bone marrow aspirate concentrate. In some embodiments, the tissue is for use in an autograft in a healthcare or veterinarian setting. In some embodiments, the tissue comprises one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma. In some embodiments, the plasma is platelet-rich plasma. In some embodiments, the tissue is an avulsed, amputated, or severed body part, or an intentionally harvested tissue. In some embodiments, the avulsed or amputated body part is one or more body parts selected from fingers, hands, forearms, arms, toes, feet, legs, ears, scalp, face, lips, penis, tongue and teeth. In some embodiments, the cells of the tissue stored in the tissue culture medium have greater viability compared to cells of a second tissue stored in saline or the University of Wisconsin Cold Storage solution (Belzer UW® Cold Storage Solution). In some embodiments, the cells of the tissue do not significantly proliferate in the tissue culture medium. In some embodiments, the healthcare or veterinarian setting is a sterile environment. In some embodiments, the healthcare setting is an organized provision of medical care to individuals, or to a community. In some embodiments, the veterinarian setting is an organized provision of medical care to animals. In some embodiments, the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

In another aspect, the disclosure provides methods of storing tissue obtained from a subject, including exposing the tissue to a composition including tissue culture medium that does not contain serum, or growth factors, and reserving the exposed tissue for re-implantation in the subject in a healthcare or veterinarian setting, wherein the tissue is stored for less than about 96 hours after obtaining the tissue from the subject. In some embodiments, the tissue culture medium does not contain peptides. In some embodiments, the composition further includes (a) a temperature-controlled packaging, or (b) a chemically-activated cool-pack. In some embodiments, the composition further includes one or more antibiotic ingredients. In some embodiments, the antibiotic ingredient is cefazolin, vancomycin, tobramycin, gentamcin, or amphotericin.

In another aspect, the disclosure provides compositions including a tissue culture storage medium, wherein the tissue culture storage medium includes the following components: water, glucose or fructose, sodium, potassium, chloride, bicarbonate, L-alanine, L-arginine, L-asparagine, L-aspartic acid, L-cysteine, L-glutamic acid, L-glutamine, glycine, L-proline, L-serine, and L-tyrosine; wherein the tissue culture storage medium does not contain serum, or growth factors. In some embodiments, the tissue culture storage medium does not contain peptides. In some embodiments, the tissue culture storage medium further including a combination of one or more of the following components: lactate, acetate, gluconate, thiamine, riboflavin, niacin, pantothenic acid, pyridoxine, biotin, folates, cobalamin, vitamin A, vitamin D, calcium, vitamin E, vitamin K, vitamin C, L-lysine, L-histidine, L-isoleucine, L-leucine, L-methionine, L-phenylalanine, L-threonine, L-tryptophan, L-valine, glutamine, taurine, glucuronolactone, carnitine, creatine, sodium pyruvate, adenine, adenosine, deoxyadenosine, guanine, guanosine, deoxyguanosine, cytosine, cytidine, deoxycytidine, thymine, thymidine, deoxythymidine, uracil, dietary fatty acids (including saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), lipoic acid, cholesterol, bisphosphonates, and/or inorganic ionic forms of elemental magnesium, calcium, boron, zinc, copper, iron, phosphorous, vanadium, strontium, silica, silicon, selenium, nickel, or manganese; wherein the composition has a near-iso-osmotic balance of 170 to 406 mOsm/L and a pH of 6.0 to 8.8 as a sterile aqueous solution. In some embodiments, the tissue culture storage medium has a pH of 5.0 to 9.0. In some embodiments, the tissue culture storage medium comprises the following components: water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.1-10 mM), sodium, potassium, chloride, and bicarbonate. In some embodiments, the tissue culture storage medium comprises the following components: water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has a near-iso-osmotic balance of 170 to 406 mOsm/L). In some embodiments, the composition further includes (a) a temperature-controlled packaging, or (b) a chemically activated cool-pack. In some embodiments, the composition further includes one or more antibiotic ingredients. In some embodiments, the antibiotic ingredient is cefazolin, vancomycin, tobramycin, gentamcin, or amphotericin.

In another aspect, the disclosure provides use of a tissue culture medium to store tissue obtained from a subject, wherein the tissue is for use in re-implantation in the subject.

In another aspect, the disclosure provides use of a composition including a sterile tissue culture medium to store tissue obtained from a subject, wherein the tissue culture medium does not contain serum or growth factors, wherein the tissue is stored for less than about 96 hours after obtaining the tissue, wherein the tissue is for use in re-implantation in the subject in a healthcare or veterinarian setting. In some embodiments, the sterile tissue culture medium does not contain peptides.

In another aspect, the disclosure provides methods of preserving cells or tissue obtained from a human subject between the time the cells or tissue are obtained from the subject and re-implantation of the cells or tissue in the subject, the method including exposing the removed cells or tissue to a tissue culture storage medium comprising water, glucose or fructose, sodium, potassium, chloride, bicarbonate, L-alanine, L-arginine, L-asparagine, L-aspartic acid, L-cysteine, L-glutamic acid, L-glutamine, glycine, L-proline, L-serine, and L-tyrosine; and wherein the tissue culture storage medium does not contain serum or growth factors. In some embodiments, the tissue culture storage medium does not contain peptides. In some embodiments, the period between obtaining and re-implantation of the cells or tissue is less than about 96 hours.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows the cell viability data of human mesenchymal stem cells (hBM-MSC) over 0-24 hours in various media as indicated. aMEM, alpha minimum essential medium; D5 1/2NS, 5% dextrose in 0.45% normal saline; UW, Belzer University of Wisconsin organ transplant saline solution.

FIG. 2 shows 100× magnification images of cell viability of human mesenchymal stem cells (hBM-MSC) at 2 hours when immersed in various media as indicated. Medium: serum-free aMEM medium.

FIG. 3 shows the cell proliferation data of human mesenchymal stem cells (hBM-MSC) over 0-120 hours in various media as indicated. Serum-free medium maintains viability without inducing a significant proliferative response.

FIG. 4 shows fractional cellular proliferation after 1 hour incubation in various media as indicated, followed by transfer to growth-factor enriched “rescue” media for 48 hours, confirming loss of cell viability in medical-grade salines relative to cell media.

### DETAILED DESCRIPTION

This disclosure is based, in part, on the findings that serum-free culture-grade media is unexpectedly superior for the storage of tissue and for use as an infusion medium and as an irrigation medium for wounds and surgical sites as compared to commonly available salines used in healthcare, which are insufficient to maintain the viability of cells and tissue. Mesenchymal stem cells, and most other human cell types, can better survive in the serum-free culture-grade media disclosed herein than in intravenous-grade salines, even for exposures less than one hour.

A detailed description of the culture-grade media for the storage of tissue and for use as an intravenous infusion medium, a wound irrigation medium, a medium for the irrigation of arthroscopic, endoscopic, microscopic, or dental procedures that require continuous irrigation, and as a medium for wound packing or vacuum devices, as well as methods of using such media, are set forth below.

### Tissue Storage Medium

Human cells or tissue intended for implantation, transplantation, infusion, or transfer into a human recipient as a human cell, tissue, and cellular and tissue-based product or HCT/P is regulated by the Center for Biologics Evaluation and Research (CBER) division of the U.S. Food & Drug Administration (FDA) and under 21 CFR Parts 1270 and 1271. Examples of such tissues are bone, skin, corneas, ligaments, tendons, dura mater, heart valves, hematopoietic stem/progenitor cells derived from peripheral and cord blood, oocytes and semen. The FDA document titled Regulatory Considerations for Human Cells, Tissues, and Cellular and Tissue-Based Products (HCT/P): Minimal Manipulation and Homologous Use requires minimal manipulation of structural tissues and processing that does not alter the original relevant characteristics of the tissue relating to the tissue's utility for reconstruction, repair, or replacement; and for cells or nonstructural tissues, processing that does not alter the relevant biological characteristics of cells or tissues. The regulation also stipulates that HCT/P is intended for homologous use only, defined as the repair, reconstruction, replacement, or supplementation of a recipient's cells or tissues with an HCT/P that performs the same basic function or functions in the recipient as in the donor.

With regards to the storage of HCT/P for homologous use, the FDA specifically stipulates that a HCT/P that is placed in a tissue medium and refrigerated, such as stored in a buffer solution; or an HCT/P that is cryopreserved and stored in liquid nitrogen vapor, would generally meet the minimal manipulation criterion. However, traditional culture media with serum and/or growth factors is not permitted in a healthcare or veterinary setting to store HCT/P due to the risks associated with altering the biological characteristics of the cells, including, but not limited to production of intracellular or cell-surface proteins and other markers of cell lineage, activation state, and proliferation, thereby altering the cells' relevant biological characteristics of multipotency and capacity for self-renewal.

The disclosure herein features a tissue culture storage medium for use in a healthcare or veterinary setting. A “tissue culture storage medium”, “tissue culture medium”, “tissue storage medium”, or a “tissue storage solution” is defined as a medium for storing cells or tissues obtained from a subject. The tissue culture storage medium can be used to preserve, store, and/or transport living biological tissues and/or populations of isolated cells temporarily prior to implantation of the living tissues or cells into a subject. Thus, the tissue culture storage medium facilitates cells and tissues to be obtained from suitable human or non-human mammalian subjects, stored for temporary periods, and transported to the site of implantation or re-implantation, all without significant loss of cell viability, biological activity, and/or tissue integrity. An important feature of the tissue culture storage medium disclosed herein is that it does not contain serum or growth factors. In some embodiments, the tissue culture storage medium disclosed herein does not include proteins (e.g., peptides greater than two amino acids in sequence). The tissue culture medium is used for the storage of cells or tissues, but not for the storage of whole organs. “Cells” refers to single cells, or a group of loosely-connected cells. “Tissue” refers to an aggregation of similar cells and their extracellular matrix from the same origin that together carry out a specific function. Tissue also refers to an aggregation of different tissues in an avulsed, amputated, or severed body part body part (e.g., a finger, toe, or limb). For example, a severed tissue can include skin, muscle, bone, nerve and blood vessels. “Whole organ” refers to a grouping of tissues into a distinct structure, such as heart, kidney, liver, etc in animals that performs a specialized task

### Infusion and Irrigation Medium

The disclosure herein features an infusion and irrigation medium for use in a healthcare or veterinary setting. A “wound irrigation medium” is defined as a medium that can be used for the steady flow of a solution across an open wound surface to achieve would hydration, to remove deeper debris, and to assist with visual examination and treatment of the wound. The wound irrigation medium can be used to remove cellular debris and surface pathogens contained in wound exudates or residue from topic applied wound care products in and around the living tissues or cells of a subject. A “surgical site irrigation medium” is defined as a medium that can be used for the steady flow of a solution across an open surgical site in a tissue of a subject, to achieve hydration of the open surgical site in a tissue of a subject, and to assist with visual examination and surgical procedures at the open surgical site in a tissue of a subject, such as in scope-assisted surgery requiring fluid insufflation. An “intravenous infusion medium” is defined as a medium that is administered through a steel needle or plastic catheter inserted in a vein of a subject, and administered at relatively slow flow rate or with a total time duration of administration that is not fixed. An intravenous infusion medium can be administered, for example, as a maintenance or resuscitation intravenous fluid for use in healthcare, field medicine, or veterinary care. This may be especially important in a trauma setting where the compositions disclosed herein may be advantageous relative to crystalloids, the latter being more dissimilar to blood or plasma compared to tissue medium compositions.

The infusion and irrigation medium can be (a) a solid that is reconstituted at the time of use with the addition of a liquid available in the healthcare environment; (b) a solid that is reconstituted at the time of use with the addition of a pre-packaged liquid; or (c) a ready-to-use liquid medium. The solid can be a block, powder, granules, etc. or in a similar form to be re-constituted at time of use into liquid media with addition of a liquid component already available in the healthcare environment (such as sterile water). The solid can be a block, powder, granules, etc. or in a similar form to be re-constituted at time of use into liquid media by mixing with a pre-packaged, ratio-correct liquid component (such as a pre-packaged jar of sterile water).

In some embodiments, the disclosure features a commercially available tissue or cell culture medium for use in an improved method for irrigating wounds or surgical sites or as an intravenous infusion. Such solutions include, but are not limited to, Eagle's minimal essential medium (EMEM); Dulbecco/Vogt's Modified Eagle's minimal essential medium (DMEM); Leibovitz's L-15 Medium (L-15); Moore's Roswell Park Memorial Institute essential medium (RPMI), RPMI-1640, and related RPMI-derived media (Cambrex); Fisher's medium, Glasgow's modified essential medium (G-MEM);

minimal essential medium (MEM); alpha minimum essential medium; aMEM; serum-free lymphocyte medium (AIM-VR) (Invitrogen Corp., Carlsbad, Calif.), Neurobasal® medium (Invitrogen Corp.), GlutaMAX® (Invitrogen Corp.), Iscove's Modified Dulbecco's Medium (IMDM) (HyClone, Logan, Utah); Human Plasma-Like Medium (HPLM) (Thermo Fisher Scientific; Waltham, MA), and other similar formulations which may be obtained from a variety of commercial suppliers including, for example, ThermoFisher (Thermo Fisher Scientific; Waltham, MA); BD (Becton, Dickinson and Company; Franklin Lakes, N.J.); Mediatech, Inc. (Herndon, Va.); Cambrex Corporation (East Rutherford, N.J.); Sigma Chemical Co. (St. Louis, Mo.); BioVeris Corp. (Salinas, Kans.); and MP Biomedicals, Inc. (Solon, Ohio). The tissue storage medium is not a parenteral solution, such as a saline, (normal saline or variations thereof), lactated Ringer's solution, iso- or near-isoosmolar glucose, plasmalyte, isolyte, or any other injectable saline prevalent in healthcare use.

In some instances, the infusion and irrigation medium further includes at least one biomembrane-sealing agent in an amount, effective to extend, lengthen, or prolong the viability of cells or tissues from a subject that are exposed to the infusion and irrigation medium during, for example, a surgical procedure.

Exemplary biomembrane-sealing agents include, but are not limited to, one or more compounds selected from the group consisting of poly(ethylene glycol) (PEG), a block copolymer containing a polyalkylene glycol, tri-block containing a polyalkylene glycol, a block copolymer containing a polyalkylene oxide, tri-block containing a polyalkylene oxide, polyvinyl alcohol, polyvinyl pyrrolidone, dextrans, hyaluronic acid, hyaluronate, poloxamine, pluronic polyols, dimethylsulfoxide, starch, HES, cellulose, sodium carboxymethyl cellulose, poly(polyethylene glycol methacrylate), poly(glycerol methacrylate), poly(glycerol acrylate), poly(polyethylene glycol acrylate), poly(alkyl oxazoline), phosphoryl choline polymers, sodium and potassium polymethacrylate, sodium and potassium polyacrylate, polymethacrylic acid and polyacrylic acid and combinations thereof. Features of biomembrane-sealing agents are disclosed in U.S. Pat. No. 9,737,071, incorporated by reference herein in its entirety.

In some instances, the infusion and irrigation medium further includes at least one antioxidant. Suitable antioxidants include, but are not limited to, one or more compounds selected from the group consisting of 2-tert-butyl-4-hydroxyanisole, 3-tert-butyl-4-hydroxyanisole, or combinations thereof (e.g., butylated hydroxyanisole, BHA); 2,6-di-tert-butyl-4-methylphenol [a.k.a., butylated hydroxytoluene (BHT)], 2,6-di-tert-butyl-p-cresol, (DBPC); ascorbic acid; ascorbate; a-tocopherol; ubiquinol-6; ubichromenol-6; α-tocopherol hydroquinone; α-tocopherol acetate; B-carotene (3,7,12,16-tetramethyl-1,18-bis(2,6,6-trimethyl-1-cyclohexenyl)-octadeca-1,3,5,7,9,11,13,15,17-nonaene); vitamin A (retinol); vitamin B1 (thiamine); vitamin B2 (riboflavin); vitamin B3 (niacin); vitamin B5 (pantothenate); vitamin B6 (pyridoxine/pyridoxal); vitamin B7 (biotin); vitamin B9 (folic acid); vitamin B10 (p-aminobenzoic acid); vitamin B12 (cobalamin/dibencozide); and one or more green tea extracts (including, but not limited to, (−) epigallocatechin-gallate; (−) gallocatechin-gallate; (−) epicatechin-gallate; (−) epigallocatechin; (+) gallocatechin; (−) epicatechin; and (−) catechin).

In some instances, the infusion and irrigation medium further includes at least one chelator, i.e., a compound that is capable of binding to one or more metal cations (including e.g., but not limited to: sodium [Na+], magnesium [Mg++], calcium [Ca++], zinc [Zn++], and iron [Fe+, Fe++, and Fe+++] ions). Exemplary chelating agents include, but are not limited to, those compounds such as deferoxamine mesylate, 2,2′-dipyridyl, and 1,10-phenanthroline, EDTA, EGTA, diaminoethane, and the like. The chelator can be present at a concentration of between about 0.001 UM and about 1 mM; e.g., between about 0.01 μM and about 100 μM, or between about 0.10 μM and about 10 μM.

In some instances, the infusion and irrigation medium further includes additional components, including, but not limited to, pharmaceutically-acceptable salts (including e.g., the acid addition salts formed with the free amino groups of a protein or peptide), organic acids (including, e.g., acetic acid, oxalic acid, tartaric acid, citric acid, malic acid, fumaric acid, mandelic acid, succinic acid, and the like). Salts formed with free carboxyl groups of amino acids can also be derived from inorganic bases such as, for example, sodium, potassium, ammonium, calcium, or ferric hydroxides, and such organic bases as isopropylamine, trimethylamine, histidine, procaine, and the like.

In some instances, the infusion and irrigation medium also includes one or more solvents, co-solvents, vehicles, diluents, buffers, growth media, storage media, carrier solutions, suspensions, colloids, and such.

In some instances, the infusion and irrigation medium also includes, one or more of a pH stabilizer, a rheological agent, a sugar (including e.g., but not limited to, allose, arabinose, atrose, cellobiose, erythrose, erythrulose, fructose, fucose, furanose, galactose, glucose, gulose, idose, inulose, lactose, lactulose, levulose, lyxose, maltose, mannose, rhaffinose, rhamnose, ribose, ribulose, sialose, sucrose, talose, threose, trehalose, xylose, and xylulose), an alcohol (including e.g., but not limited to, ethanol, propanol, isopropanol, and PVA), a sugar alcohol (including e.g., but not limited to: adonitol, arabitol, dulcitol, erythritol, inositol, isomalt, lacitol, maltitol, mannitol, sorbitol, and xylitol), an inorganic salt (including e.g., but not limited to: NaCl, NaPO4, CaPO4, KCl, etc.), lycopene, proanthocyanidin, a surfactant, a wetting agent, an osmotic agent, or an antifoam compound.

In some instances, the infusion and irrigation medium also includes one or more compounds including, but not limited to: organic acids (including, e.g., but not limited to: citric acid, malic acid, succinic acid), starches (including, e.g., but not limited to: hydroxyethyl starch [HES]), vitamins, hormones (e.g., insulin), a pro-drug, a nuclease inhibitor, a kinase inhibitor, an antimicrobial agent (including, but not limited to:

microbicides, mildewcides, fungicides, bactericides, viricides, antimycotics, antihelminths, etc).

The infusion and irrigation medium of the disclosure may also optionally contain one or more compounds involved in maintaining cellular function, preserving cellular integrity, or maintaining the differentiated state of a given population of cells.

In one example, the infusion and irrigation medium includes, but is not limited to, the following components: water, glucose or fructose, sodium, potassium, chloride, bicarbonate, and at least one amino acid. In addition, the infusion and irrigation medium may further include, but is not limited to, a combination of one or more of the following components: lactate, galactose, acetate, gluconate, thiamine, riboflavin, niacin, pantothenic acid, para-aminobenzoic acid, pyridoxine, biotin, folates, cobalamin, inositol, vitamin A, vitamin D, vitamin E, vitamin K, vitamin C, L-alanine, L-arginine, L-asparagine, L-aspartic acid, L-cysteine, L-cystine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, taurine, glutathione, glucuronolactone, acetone, carnitine, creatine, creatinine, glycerol, hypoxanthine, sodium pyruvate, urea, uridine, acetylcarnitine, betaine, L-amino-N-butyric acid, L-citrulline, L-hydroxyproline, L-ornithine, N-acetylglycine, 3-hydroxybutyric acid, alpha ketoglutarate, citric acid, formic acid, malic acid, malonic acid, succinic acid, uric acid adenine, adenosine, deoxyadenosine, guanine, guanosine, deoxyguanosine, cytosine, cytidine, deoxycytidine, thymine, thymidine, deoxythymidine, uracil, dietary fatty acids (including saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), lipoic acid, cholesterol, bisphosphonates, ammonium, sulfate, chloride, phosphate, bicarbonate, nitrate, and/or inorganic ionic forms of elemental magnesium, calcium, boron, zinc, copper, iron, phosphorous, vanadium, strontium, silica, silicon, selenium, nickel, or manganese; wherein the composition has a near-iso-osmotic balance of 170 to 406 mOsm/L and a pH of 5.0 to 9.0 as a sterile aqueous solution.

In some embodiments, the infusion and irrigation medium includes, but is not limited to the following components: water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.01-10 mM), sodium, potassium, chloride, and bicarbonate. In some embodiments, the infusion and irrigation medium includes, but is not limited to the following components: water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has an near-iso-osmotic balance of 170 to 406 mOsm/L). In some embodiments, the infusion and irrigation “minimum medium” consists essentially of water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.01-10 mM), sodium, potassium, chloride, and bicarbonate. In some embodiments, the infusion and irrigation “minimum medium” consists essentially of water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has a near-iso-osmotic balance of 170 to 406 mOsm/L).

### Compositions of Tissue Storage, Infusion, and Irrigation Medium

The disclosure herein features a composition comprising the disclosed tissue storage, infusion, and irrigation medium. The composition may include additional components, such as temperature-controlled packaging, or a chemically-activated cool-pack. The composition may further include one or more antibiotic ingredients. Antibiotic ingredients include but are not limited to cefazolin, vancomycin, tobramycin, gentamcin, or amphotericin.

The temperature-controlled packaging permits maintenance of a stable temperature to prevent exposure of the tissue to excessive heat or cold/freezing temperatures. In some embodiments, the temperature-controlled packaging permits a gradual cooling of the tissue (to 6° C. or higher) when the composition containing the tissue is placed in a cooling device. In some embodiments, the temperature-controlled packaging permits a gradual warming of the tissue (to 6° C. or higher) when the composition containing the tissue is placed in a warming device.

The compositions of the disclosure are formulated as “end-user,” “active” or “working” solutions, or alternatively, prepared in the form of one or more concentrated “stock” solutions that may subsequently be diluted into an appropriate solvent, or buffer, to prepare final “working” solutions. Stock solutions may then be subsequently diluted five-, ten-, or one-hundred-100-fold, respectively, by an end-user into a suitable biological buffer, commercially-available organ transport solution, or growth medium to produce the final “working” solutions with each of the ingredients in the correct amount for use by the practitioner.

Alternatively, the compositions of the disclosure may be formulated for packaging and/or commercial sale as a ready-to-use solution, either alone, or optionally supplemented with one or more various additional ingredients as enumerated herein, and may be sterilized or pasteurized prior to sale or use using one of the conventional fluid sterilization/pasteurization means known in the art, such as for example, by autoclaving, irradiation, ultrafiltration, or such like.

In some embodiments, the tissue storage, irrigation, and infusion medium is a composition similar to that of extra-cellular fluid (ECF) found in the human body. ECF typically includes nutritive ingredients including sugar, salts, amino acids, and vitamins. These nutritive ingredients permit function of the major metabolic pathways required for at least temporary cell survival, including glycolysis, the citric acid cycle, and the urea cycle. ECF and human plasma also include proteinaceous components and growth factors that drive cellular signaling and participate in maintaining systemic homeostasis in addition to maintaining local tissue homeostasis provided by the basic nutritive ingredients.

ECF in a normal physiologic state consists of an ultrafiltrate of human blood plasma. Therefore, the composition of ECF differs from that of human plasma, while maintaining physiologic pH, ionic concentrations, and osmolarity of the extra-cellular environment. This permits maintenance of healthy cellular membrane potentials and baseline cellular metabolism, so as to prevent cell stress responses that contribute to pathologic states. ECF and human plasma are more complex than currently available medical salines used for tissue preservation, irrigation, and infusion in healthcare. As exemplified in further detail below, musculoskeletal progenitor cells (human mesenchymal stromal or stem cells) grow and respond poorly in salines used in healthcare settings. Salines consist of sugars and salts in non-physiologic concentrations.

While not wishing to be bound by theory, the lack of complexity, as well as non-physiologic pH, ionic concentrations, and osmolarity, contribute to poor tissue response to medical-grade salines.

The tissue storage, irrigation, and infusion medium compositions disclosed herein comprise water, glucose or fructose, sodium, potassium, chloride, and at least one amino acid, provided at a physiologic or near-physiologic pH and osmolarity so as to maintain cellular homeostasis for a limited period of time. The tissue storage, irrigation, and infusion medium compositions disclosed herein are an improvement for tissue preservation, irrigation, and infusion in healthcare and veterinary settings relative to currently available medical-grade salines.

In some embodiments, the compositions may be formulated as sterile solutions, or alternatively, as sterilized powders by lyophilization or freeze-drying of the formulations using conventional methodologies. In the case of compositions that comprise large MW biomembrane sealing agents that are un-amenable to heat sterilization, it may be necessary to formulate the final working compositions from a powder or solid of the active ingredient, which may be later reconstituted and or filter sterilized or irradiated to form the final sterile composition.

In some embodiments, the compositions disclosed herein do not include serum. In some embodiments, the compositions disclosed herein do not include serum because serum is typically obtained by human or xenogeneic blood donation. For these processes, there can be batch variability in compositions of sera and present quality control and consistency challenges. Human and xenogeneic sera may also introduce biological contaminants into the compositions.

In some embodiments, the compositions herein do not include growth factors. In some embodiments, the compositions disclosed herein do not include growth factors because the compositions are formulated to maintain cellular homeostasis without inducing substantial phenotypic or proliferative changes in cells and/or tissues. In some embodiments, the compositions disclosed herein do not include growth factors in order to avoid unwanted or off-target cellular signaling.

In some embodiments, the compositions herein do not include peptides. In some embodiments, the compositions disclosed herein do not include peptides because the compositions are formulated to maintain cellular homeostasis without inducing substantial phenotypic or proliferative changes in cells and/or tissues. In some embodiments, the compositions disclosed herein do not include peptides in order to avoid unwanted or off-target cellular signaling.

The final storage/working solutions may be mixed aseptically at time of use, pre-mixed prior to sale and delivered as a ready-to-use, or ready-to-dilute product, or, alternatively, the solution or plurality of individual reagent solutions delivered as is, which are then reconstituted/mixed under non-sterile conditions by the end-user, and then at time sterile filtered immediately prior to use.

By using the compositions described herein, the cells or tissues at the site of infusion or irrigation, for example at a surgical site, a trauma-associated wound, or vascular endothelial tissue, remain substantially viable, and as such, retain substantial biologic activity, while contacted by the infusion or irrigation medium, or stored in, and/or transported in, such a composition, for periods that are significantly longer than those afforded by either commercially-available buffers, biological storage and/or organ and tissue transport/storage solutions, or intravenous-grade saline solutions or other intravenous infusion media found in the prior art.

The compositions disclosed herein will find particular use in the storage and/or transport of tissues and as an intravenous infusion. When used in the storage and/or transport of tissues, an ambient storage temperature of about 6° C. or higher can be used (such as 6° C. to about 40° C.). “About” refers to the numerical value±10%. While it is contemplated that slight variation in temperature during the storage/transport process will not adversely affect the short-term integrity, biological function, or cellular viability of the stored tissue or cells, the material will be maintained and transported under environmental conditions of about 6° C. to about 40° C.

### Methods of Storing Cells and Tissues

The disclosure herein features a method of storing tissue obtained from a subject in the disclosed tissue culture medium, which includes the steps of exposing the tissue to the disclosed tissue culture medium that does not contain serum, peptides, or growth factors; and reserving the exposed tissue for re-implantation in the subject. The tissue is stored for less than about 96 hours after obtaining the tissue from the subject.

The cells or tissues to be stored using the compositions of this disclosure are obtained from a subject in a healthcare or veterinary setting. A “subject” is defined herein as a human or a non-human mammal. Non-human mammals include but are not limited to, cattle, horses, sheep, pigs, goats, rabbits, dogs, cats, and non-human primates. As used herein, the term “cells or tissues”, “tissue”, or “tissue” refer to live non-whole organ cells or tissues, that continue to maintain their biological functions while in the tissue culture medium. The tissue can be a structural tissue or a non-structural (cellular) tissue. In some embodiments, the tissue is a stem cell-containing tissue or a cell fraction. The stem cell-containing tissue is bone marrow aspirate or bone marrow aspirate concentrate. In some embodiments, the tissue is for use in an autograft in a healthcare or veterinarian setting. Autograft refers to a subject's own tissue which is re-implanted into the subject. The tissue to be stored can be any tissue, including, but not limited to nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma (such as platelet-rich plasma).

In some cases, the tissue is an avulsed, amputated, or severed body part, or an intentionally harvested tissue. For instance, the avulsed or amputated body part is a body part including but not limited to fingers, hands, forearms, arms, toes, feet, legs, ears, scalp, face, lips, penis, tongue and teeth.

In some cases, a first population of cells or a first tissue stored in the tissue culture medium has greater viability compared to a second population of cells or a second tissue stored in saline or the University of Wisconsin Cold Storage solution (Belzer UW® Cold Storage Solution.

In some embodiments, the cells of the tissue do not proliferate in the tissue culture medium. Cells that “do not proliferate” are cells that are unable to actively divide while stored in the tissue culture medium of the disclosure, or cells that minimally or insignificantly proliferate.

The present disclosure provides a population of cells, or an explanted biological tissue that is prepared by methods known in the art. Although there is no inherent limitations to the cells, or tissues, that may benefit from being maintained and/or transported in one or more of the disclosed storage compositions, it is contemplated that in most circumstances, the explanted population of the subject's cells, or tissue(s), to be maintained will generally be of animal origin, and in particular, of mammalian origin. Exemplary donor cells, or tissues, include, but are not limited to, those of human, bovine, ovine, porcine, equine, canine, feline, caprine, luprine, or non-human primate origin. In certain embodiments, the human may be a patient under the care of a physician or other medical professional, and is, will, or may have been in need of transplantation or one or more cells, or tissues, harvested from a suitable donor mammal. In some embodiments, the human may be a patient under the care of a physician or other medical professional, and is, will, or may have been in need of implantation or one or more cells or tissues, obtained from a the same human patient, e.g., storing avulsed fingers, knocked-out teeth, etc. in an emergency medical response setting, or temporary intra-operative storage of explanted tissue grafts or cell fractions. The term “obtained from” a subject refers to intentionally harvested tissue or unintentionally acquired tissue (such as an avulsed body part).

The disclosure herein features a method of storing tissue obtained from a subject in a tissue culture medium of the disclosure. The tissue is stored for less than about 96 hours (e.g., about an hour, about 2 hours, about 3 hours, about 4 hours, about 6 hours, about 8 hours, about 10 hours, about 15 hours, about 20 hours, about 25 hours, about 30 hours, about 36 hours, about 48 hours, about 60 hours, about 72 hours, about 84 hours, or about 96 hours) after obtaining the tissue from the subject, and is for use in re-implantation in the subject in a healthcare or veterinarian setting.

In some embodiments, the tissue is for use in implantation in the subject from whom the tissue was obtained. In some embodiments, the tissue is for use in allografts or xenografts. In some applications of the present methods, the recipient animals will be mammalian species, such as livestock, animals under veterinary care, and most often, humans. Particularly desirable donor animals include, but are not limited to, those mammals from which cells, or tissues, may be harvested that are suitable for transplantation into a human recipient. These may include, but are not limited to cows, dogs, goats; horses, cats, chickens, humans, rabbits, hares, wolves, mice, rats, sheep, pigs, foxes, non-human primates, or other mammalian species from which cells or tissues may be harvested.

Methods of Infusing and/or Irrigating Tissues

### Intravenous Infusion

In some embodiments, the compositions disclosed herein are used as an intravenous infusion. For example, the compositions disclosed herein can be used as a maintenance or resuscitation intravenous fluid for use in healthcare, field medicine, or veterinary care. The compositions disclosed herein can be used as an intravenous infusion in a trauma setting. The compositions disclosed herein can be used for the management of critically hypotensive patients to improve intravascular volume. In some embodiments, due to disruption of the integrity of the endothelial glycocalyx layer under inflammatory conditions, such as sepsis, surgery, trauma, or traumatic brain injury, the compositions disclosed herein are more effective and safer than alternative intravenous infusion media, for example, physiologic saline, crystalloids, colloids, or mineral salts. In some embodiments, patients receiving intravenous administration of the compositions disclosed herein exhibit reduced all-cause mortality rate compared to patients receiving alternative intravenous infusion media, for example, physiologic saline, crystalloids, colloids, or mineral salts. In some embodiments, patients receiving intravenous administration of the compositions disclosed herein exhibit reduced kidney injury compared to patients receiving alternative intravenous infusion media, for example, physiologic saline, crystalloids, colloids, or mineral salts. In some embodiments, patients receiving intravenous administration of the compositions disclosed herein exhibit reduced intimal tissue damage compared to patients receiving alternative intravenous infusion media, for example, physiologic saline, crystalloids, colloids, or mineral salts. In some embodiments, the compositions disclosed herein for use as an intravenous infusion are not crystalloids, plasmalyte, isolyte, lactated ringers, or sodium chloride. In some embodiments, the compositions disclosed herein for use as an intravenous infusion do not comprise albumin, donor plasma, donor serum, donor platelets, or donor whole blood, or any other colloids.

### Irrigation During Surgical Procedures

In some embodiments, the compositions disclosed herein are used as an irrigant for arthroscopic, endoscopic, microscopic, or dental procedures, or any medical procedure that requires continuous irrigation. For example, the compositions disclosed herein can be used as an insufflation fluid for use in joint arthroscopic procedures, where prolonged exposure to certain irrigants may incur tissue damage. When used as an irrigant for surgical procedures, the compositions disclosed herein can promote the viability of cells and tissues exposed to the irrigant. In some embodiments, use of the compositions disclosed herein for surgical site irrigation results in reduced tissue damage at the surgical site compared to use of other surgical site irrigation media, for example, physiologic saline, crystalloids, colloids, or mineral salts.

### Wound Irrigation

In some embodiments, the compositions disclosed herein are used as a wound irrigation fluid, for example, during emergency medical response for the purposes of washing a contaminated wound. When used as a wound irrigation fluid, the compositions disclosed herein can promote the viability of cells and tissues exposed to the irrigant during wound care. In some embodiments, use of the compositions disclosed herein for wound irrigation results in reduced tissue damage at the wound site compared to use of other wound irrigation media, for example, physiologic saline, crystalloids, colloids, or mineral salts.

### Wound Packing

In some embodiments, the compositions disclosed herein are used for wound packing and in wound irrigation/vacuum devices. In some embodiments, the compositions disclosed herein are used for continuous fluid flow vacuum-assisted closure (VAC) of wounds to improve tissue viability. When used for continuous fluid flow VAC, the compositions disclosed herein can promote the viability of cells and tissues exposed to the fluid during wound care. In some embodiments, use of the compositions disclosed herein for continuous fluid flow VAC results in reduced tissue damage at the wound site compared to use of other fluids used for continuous fluid flow VAC, for example, physiologic saline, crystalloids, colloids, or mineral salts. In some embodiments, the compositions disclosed herein are used for wet-to-dry wound packing to promote accelerated wound healing. When used for wet-to-dry wound packing, the compositions disclosed herein can promote the viability of cells and tissues exposed to the fluid during wound care. In some embodiments, use of the compositions disclosed herein for wet-to-dry wound packing results in reduced tissue damage at the wound site compared to use of other fluids used for continuous fluid flow VAC, for example, physiologic saline, crystalloids, colloids, or mineral salts.

In some embodiments, the compositions disclosed herein are used as a wound irrigation medium, a surgical site irrigation medium, a wound packing medium, or an intravenous infusion medium in a healthcare or veterinarian setting. In some embodiments, the storage of cells and tissues is for use in an autograft in a healthcare or veterinarian setting. The healthcare or veterinarian setting is a sterile, semi-sterile, or non-sterile environment. An example of a “sterile” environment is a hospital operating room surgical field. An example of a “semi-sterile” environment is an ambulance responding to an accident site or one tending to a severed body part while employing medical sterile technique. An example of a “non-sterile” environment is in the military field. Semi-sterile and/or non-sterile environments may not be sterile but are still clean-contaminated.

A healthcare setting refers to an organized provision of medical care to individuals, or to a community. A healthcare or veterinary setting is any setting involving the surgical or procedural intervention that involves treatment of an illness or injury in a sterile, semi-sterile, or non-sterile emergency manner. The methods described herein can be used by a number of healthcare workers, such as those defined by the World Health Organization. See Classification of health workforce statistics, World Health Organization Geneva. Healthcare worker includes, but is not limited to, general medical practitioners, specialist medical practitioners, nursing professionals, midwifery professionals, traditional and complementary medicine professionals, paramedical professionals, dentists, pharmacists, environmental and occupational health and hygiene professionals, therapeutic equipment technicians, medical laboratory technicians, pharmaceutical assistants, prosthetic technicians, nursing associates, midwifery associates, traditional and complementary medicine associates, dental assistants, community health workers, medical assistants, ambulance workers, health care assistants, home-based personal care workers, life science professionals, life science technicians, armed forces medical occupations, medical students, or hospital volunteers.

A healthcare or veterinary setting includes in the inpatient hospital setting, outpatient setting, operating room, doctor's office, veterinarian office, field office, procedural suite, nursing office, mass sporting event, and in situations requiring emergency medical response, including ambulance response and field medical response in a military field office.

### Cellular/Tissue Viability and Proliferation Assays

The viability and proliferation of cells and tissues, particularly those obtained from humans and non-human mammals, can be determined by several published assays that are known to those of skill in the relevant art. In one assay, the viability of cells is readily determined by using a microscopic assay that is commonly referred to in the art as a “live/dead assay” (Huntley J S, et al., J. Bone Joint Surg. Am., 87 (2): 351-60, February 2005). In one such assay, the biologic dyes 5-chloromethylfluorescein diacetate and propidium iodide (which differentially stain living and non-living cells) are employed and evaluated by a microscopy-based assay. Such dyes are typically fluorescent, and the fluorescence may be detected and used to produce dual-parameter fluorescence histograms, most typically using fluorescence microscopy techniques to distinguish the living vs. the non-living cells, in which the living and non-living cells each fluoresce at distinctly-different wavelengths.

To determine the % viability of tissues that have been exposed to the composition disclosed herein, a biological sample may be initially assayed for viability (typically within 48 hours of harvest from the donor animal) to determine an “initial viability.” Subsequent viability determinations are then made on the tissue over a period of time to determine “current viability.” The % viability can therefore be determined at any time post-harvest using the following equation:

$$[ ( current viability ) / ( initial viability ) ] \times 100 = Percent viability$$

If desired, multiple samples may be analyzed and averaged both at initial assay, and/or during subsequent analyses to determine an “average viability” of the harvested tissue. In addition to the live/dead cell staining described herein, the viability of cells, or tissues, may be determined by one or more of a variety of well-known biological viability assays, including for example, by quantitation of 35SO4-uptake (Pennock A T, et al., J. Knee Surg., 19 (4): 265-72, October 2006), or by glycosaminoglycan (GAG) quantitation (Pennock A T, et al., J. Knee Surg., 19 (4): 265-72, October 2006) to name only a few.

Alternatively, the determination of tissue, or cell, viability may also include one or more biochemical or anatomical assays that are known in the art, and which provide qualitative and/or quantitation evidence of the biological activity or functionality of the explanted tissue once it is introduced into the recipient animal.

Various cell proliferation assays are known in the art and may be employed to determine the quality of the stored cells or tissues. These include, but are not limited to tryphan blue, MTT assay, XTT assay, MTS assay, Water Soluble Tetrazolium Assay, Alamar Blue, and others described in Adan A et al., Current Pharmaceutical Biotechnology, 2016, 17, 1213-1221.

### EXAMPLES

The practice of the methods and compositions of the disclosure employs, unless otherwise indicated, conventional techniques of cell culture, which are well within the purview of the skilled artisan. The invention, as described in the following examples, do not limit the scope of the invention described in the claims.

### Example 1: HBM-MSCs Survive Better in Culture Medium (Alpha Minimum Essential Medium; aMEM) than in Medical-Grade Salines

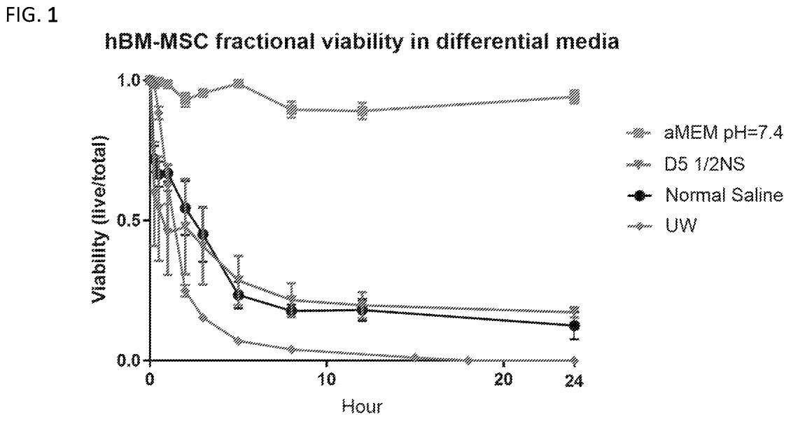

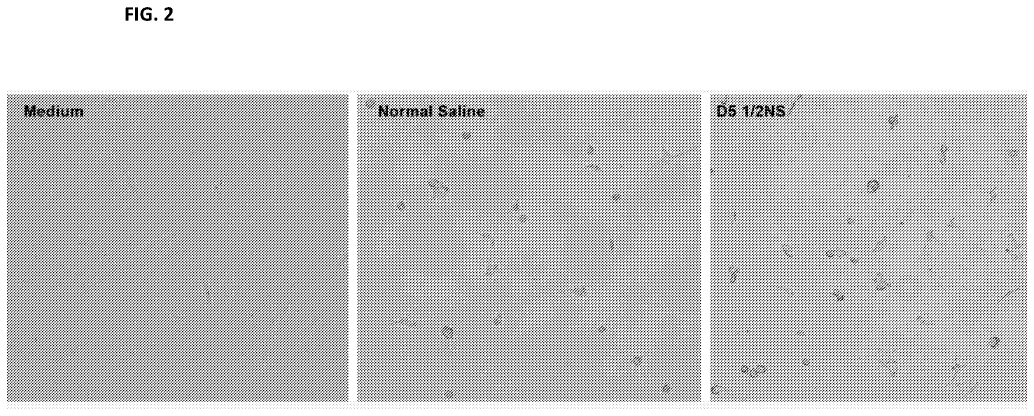

Human mesenchymal stem cells (hBM-MSC; a common progenitor cell found in musculoskeletal tissues; American Type Culture Collection (ATCC), Manassas, VA) were placed in 3 different media-alpha minimum essential medium (aMEM; Gibco, Thermo Fisher Scientific, Waltham, MA; pH 7.4), normal saline (0.9% sodium chloride; B. Braun, Melsungen, Germany), and 5% dextrose half-saline (D5 1/2NS; B. Braun, Melsungen, Germany). Cells were seeded in 6-well tissue culture dishes (Costar, Corning, Corning, NY) as a 10% confluence monolayer. The viability of the cells was determined at various timepoints over the course of 24 hours by light microscopic comparative counting of cellular pyknosis-a high-threshold method of determining severe irreversible cell damage. Biological triplicates were performed for each measurement to determine statistical variance. As shown in FIG. 1, in vitro cell viability data demonstrates that hBM-MSC can better survive in culture medium (alpha minimum essential medium; aMEM) than in medical-grade salines available for use in healthcare (5% dextrose half-saline, D5 1/2NS; and Belzer University of Wisconsin organ transplant saline solution, UW). Significant cell death occurs within the first 30 minutes of cellular exposure to medical grade salines. However, there is negligible cell death over the course of 24 hours of cells exposed to aMEM media. As shown in FIG. 2, at 2 hours, cells remain healthy and phenotypically normal in aMEM, whereas near-complete cell death has occurred in normal saline or D5 1/2NS. These results indicate that the use of culture-grade media is superior in terms of cell viability for the storage of tissue as compared to commonly available salines used in healthcare. These results also suggest that iso-osmolarity or glucose alone cannot significantly sustain cell viability.

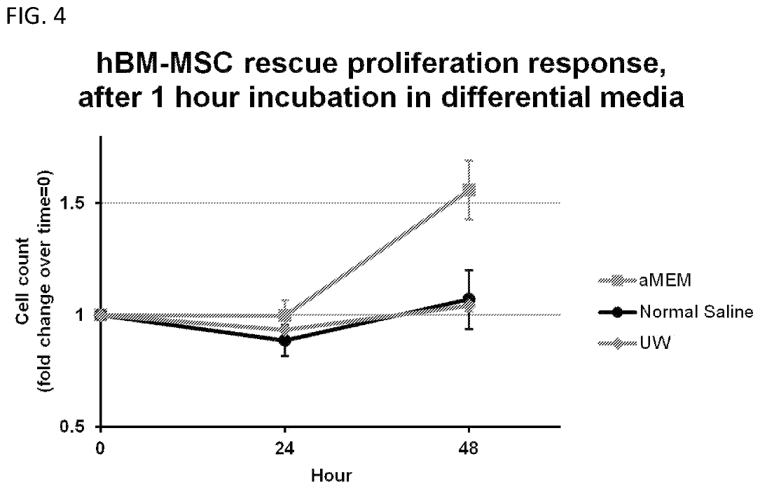

Further, as shown in FIG. 4, after a 1 hour incubation in each of aMEM, saline, and Belzer UW medium, hBM-MSCs successfully proliferated upon rescue after aMEM incubation but not after incubation in saline or Belzer UW medium. These results indicate that aMEM promotes cell viability and suggest that aMEM is more suitable as a tissue storage, irrigation, and infusion medium than are saline or Belzer UW medium.

### Example 2: HBM-MSCs do not Proliferate in Serum-Free Culture Medium Compared to Serum-Enriched Culture Medium

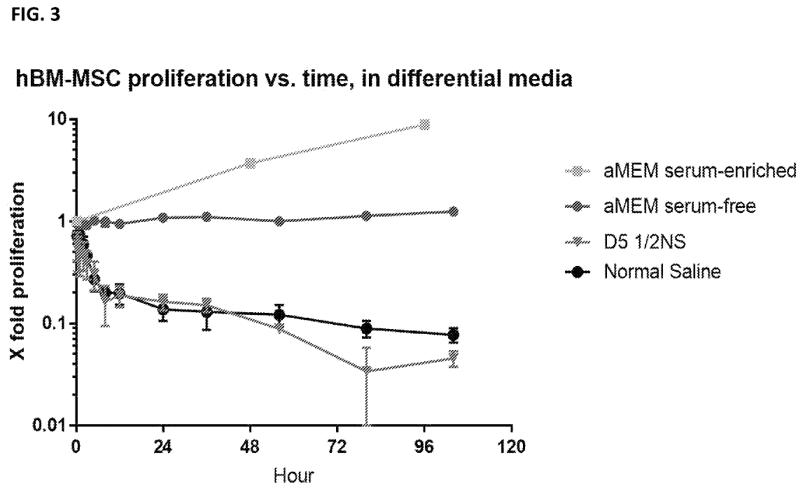

hBM-MSCs were placed in 4 different media-serum-enriched (Fetal Bovine Serum; Atlanta Biologicals, Flowery Branch, GA) alpha minimum essential medium (aMEM; Gibco, Thermo Fisher Scientific, Waltham, MA), aMEM (serum-free), normal saline (0.9% sodium chloride; B. Braun, Melsungen, Germany), and 5% dextrose half-saline (D5 1/2NS; B. Braun, Melsungen, Germany). The fold proliferation of the cells was determined by manual light microscopic cell counting at various timepoints over the course of 24 hours. Biological triplicates were performed for each measurement to determine statistical variance. As shown in FIG. 3, in vitro cell proliferation data demonstrates that hBM-MSCs maintain viability in the serum-free environment. However, in a serum-enriched environment, hBM-MSCs undergo profound proliferation during the first few days of cell culture. These results indicate that serum-free medium can allow for cellular persistence, with minimal cell proliferation.

### Example 3: Use of Tissue Storage Medium in Spinal Fusion Surgery

In one example, a surgeon would use the tissue medium during the course of a spinal fusion surgery. Spinal fusion surgery often requires the use of autograft (i.e., a patient's own tissue) bone. The liquid tissue medium would be provided in a sterile manner and made available to the surgeon on the sterile field. This can take the form of a sterile medical jar that contains shelf-stable, pre-prepared liquid tissue medium, in various available volumes appropriate for surgical use (for example, 50 cc, 150 cc, and 500 cc jars). The surgeon would then temporarily store harvested bone, which contains live cells, in this tissue medium until ready for re-implantation. The tissue medium will keep the cells within the bone graft alive. A few hours later, when the surgical site has been made ready for grafting, the surgeon would remove the bone graft from the tissue medium and place the live bone graft back in the body of the patient.

### Example 4: Use of Tissue Storage Medium in Staged Surgical Procedure

In one example, a surgeon may temporarily store autograft tissue outside of the body during a staged surgical procedure that spans multiple sessions. For example, in an emergency situation, a patient's index spinal fusion surgery may be cancelled due to hemodynamic instability but the bone graft has already been harvested. This bone graft may then be stored in the tissue medium until the patient is able to return to the operating room a few days later.

### Example 5: Use of Tissue Storage Medium for Patients in Auto Accidents

In one example, upon arrival to an auto accident, an ambulance would carry a small store of shelf-stable, pre-prepared liquid tissue medium for immediate availability. A paramedic or emergency medical technician would be able to store a severed body part (such as a tooth, or finger) in the sterile liquid tissue medium. A specific composition of the tissue medium made appropriate for use in this setting may be used, which may in one instance include special packaging of the tissue medium that permits easy handling and temperature control (e.g., packaging that permits a 1 degree Celsius per minute drop in temperature when stored in the refrigerator, which can improve tissue viability), or in another instance may include optionally additional antibiotic ingredients, such as cefazolin, gentamcin, and amphotericin, to prevent contamination of the tissue. The body part may be stored in this medium until it is re-attached by a medical professional.

### Example 6: Use of Tissue Storage Medium for Patients on the Battlefield

In one example, on the battlefield, severed body parts may be stored in a similar way as done in Example 5, with additional packaging composition to allow immediate tissue cooling without the need for additional refrigeration equipment (such as a chemically activated cool-pack) to prolong cell viability.

### Example 7: Use of Surgical Site Irrigation Medium in Hip Replacement Surgery

In one example, a surgeon would use the surgical site irrigation medium during a total hip arthroplasty (THA) procedure. THA surgery often requires continuous irrigation of the surgical site. The surgical site irrigation medium would be provided in a sterile manner and made available to the surgeon on the sterile field. This can take the form of a sterile medical jar that contains shelf-stable, pre-prepared liquid surgical site irrigation medium, in various available volumes appropriate for surgical use (for example, 50 cc, 150 cc, and 500 cc jars). The surgeon would perform intraoperative irrigation with the surgical site irrigation medium as necessary throughout the course of the operation.

### Example 8: Use of Intravenous Infusion Medium for Trauma Patients

In one example, upon arrival to an auto accident, an ambulance would carry a supply of shelf-stable, pre-prepared liquid intravenous infusion medium for immediate availability. A paramedic or emergency medical technician would be able to administer intravenous infusion medium to a patient experiencing trauma, for example, a patient with severe blood loss and shock after an auto accident. Trauma and acute blood loss trigger compensatory mechanisms aimed at restoring volume deficits to maintain adequate perfusion of vital organs. Rapid administration of the intravenous infusion medium can stabilize homeostasis and replace the fluid lost from the interstitial compartment to the intravascular spaces, while minimizing injury to the vascular endothelial tissues.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of irrigating a tissue of a subject, the method comprising exposing the tissue to a liquid medium that does not contain serum or growth factors.

2. The method of claim 1, wherein the liquid medium does not contain peptides.

3. The method of claim 1 or 2, wherein the tissue is at a site of a wound in a subject.

4. The method of any one of claim 2, wherein the site of the wound has been treated with a wound packing and/or vacuum device.

5. The method of claim 1, wherein the tissue is at a surgical site in a subject.

6. The method of claim 5, wherein the surgical site is an arthroscopic surgical site, an endoscopic surgical site, a microscopic surgical site, or a dental surgical site.

7. The method of any of claims 1-6, wherein the subject is a human or a non-human mammal.

8. The method of any one of claims 1-7, wherein the infusion and/or irrigation medium is Dulbecco's Modified Eagle Medium (DMEM) or alpha-minimum essential medium (aMEM) or similar compositions thereof.

9. The method of any one of claims 1-8, wherein the infusion and/or irrigation medium is a sterile cell culture medium.

10. The method of any one of claims 1-7, wherein the infusion and/or irrigation medium has a similar composition to human plasma or human plasma-like medium (HPLM).

11. The method of any one of claims 1-10, wherein the infusion and/or irrigation medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium.

12. The method of any one of claims 1-11, wherein the infusion and/or irrigation medium further comprises one or more antibiotic ingredients.

13. The method of claim 12, wherein the antibiotic ingredient is cefazolin, vancomycin, tobramycin, gentamicin, or amphotericin.

14. The method of any one of claims 1-13, wherein the tissue comprises one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma.

15. The method of any one of claims 1-14, wherein the cells of the tissue contacted by the infusion and/or irrigation medium have greater viability compared to cells of a second tissue contacted by an intravenous-grade saline solution.

16. The method of any one of claims 1-15, wherein the healthcare or veterinarian setting is a sterile environment.

17. The method of any one of claims 1-16, wherein the healthcare setting is an organized provision of medical care to individuals, or to a community.

18. The method of any one of claims 1-16, wherein the veterinarian setting is an organized provision of medical care to animals.

19. The method of any one of claims 1-18, wherein the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

20. A method of providing an intravenous infusion to a subject, comprising intravenously providing a composition comprising tissue culture medium that does not contain serum, or growth factors.

21. The method of claim 20, wherein the composition does not contain peptides.

22. The method of claim 20 or 21, wherein the composition further comprises one or more antibiotic ingredients.

23. The method of claim 22, wherein the antibiotic ingredient is cefazolin, vancomycin, tobramycin, gentamicin, or amphotericin.

24. A composition comprising an infusion and/or irrigation medium, wherein the infusion and/or irrigation medium comprises the following components: water, glucose or fructose, sodium, potassium, chloride, bicarbonate, and at least one amino acid; wherein the infusion and/or irrigation medium does not contain serum or growth factors.

25. The composition of claim 24, wherein the infusion and/or irrigation medium does not contain peptides.

26. The composition of claim 24 or 25, wherein the infusion and/or irrigation medium further comprises a combination of one or more of the following components: lactate, galactose, acetate, gluconate, thiamine, riboflavin, niacin, pantothenic acid, para-aminobenzoic acid, pyridoxine, biotin, folates, cobalamin, inositol, vitamin A, vitamin D, vitamin E, vitamin K, vitamin C, L-alanine, L-arginine, L-asparagine, L-aspartic acid, L-cysteine, L-cystine, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, taurine, glutathione, glucuronolactone, acetone, carnitine, creatine, creatinine, glycerol, hypoxanthine, sodium pyruvate, urea, uridine, acetylcarnitine, betaine, L-amino-N-butyric acid, L-citrulline, L-hydroxyproline, L-ornithine, N-acetylglycine, 3-hydroxybutyric acid, alpha ketoglutarate, citric acid, formic acid, malic acid, malonic acid, succinic acid, uric acid adenine, adenosine, deoxyadenosine, guanine, guanosine, deoxyguanosine, cytosine, cytidine, deoxycytidine, thymine, thymidine, deoxythymidine, uracil, dietary fatty acids (including saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), lipoic acid, cholesterol, bisphosphonates, ammonium, sulfate, chloride, phosphate, bicarbonate, nitrate, and/or inorganic ionic forms of elemental magnesium, calcium, boron, zinc, copper, iron, phosphorous, vanadium, strontium, silica, silicon, selenium, nickel, or manganese; wherein the composition has a near-iso-osmotic balance of 170 to 406 mOsm/L and a pH of 6.0 to 8.8 as a sterile aqueous solution.

27. The composition of any one of claims 24-26, wherein the infusion and/or irrigation medium has a pH of 5.0 to 9.0.

28. The composition of any one of claims 24-27, wherein the infusion and/or irrigation medium further comprises the following components: water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.1-10 mM), sodium, potassium, chloride, and bicarbonate.

29. The composition of any one of claims 24-28, wherein the infusion and/or irrigation medium comprises the following components: water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has a near-iso-osmotic balance of 170 to 406 mOsm/L).

30. The composition of any one of claims 24-29, wherein the composition further comprises one or more antibiotic ingredients.

31. The composition of claim 30, wherein the antibiotic ingredient is cefazolin, vancomycin, tobramycin, gentamicin, or amphotericin.

32. A method of irrigating a tissue of a subject, the method comprising exposing the tissue to a liquid medium that does not contain serum or growth factors.

33. The method of claim 32, wherein the tissue is at the site of a wound in a subject.

34. The method of claim 33, wherein the site of the wound has been treated with a wound packing and/or vacuum device.

35. The method of claim 32, wherein the tissue is at a surgical site in a subject.

36. The method of claim 35, wherein the surgical site is an arthroscopic surgical site, an endoscopic surgical site, a microscopic surgical site, or a dental surgical site.

37. The method of any of claims 32-36, wherein the subject is a human or a non-human mammal.

38. The method of any one of claims 32-37, wherein the infusion and/or irrigation medium is Dulbecco's Modified Eagle Medium (DMEM) or alpha-minimum essential medium (αMEM) or similar compositions thereof.

39. The method of any one of claims 32-38, wherein the infusion and/or irrigation medium is a sterile cell culture medium.

40. The method of any one of claims 32-38, wherein the infusion and/or irrigation medium has a similar composition to human plasma or human plasma-like medium (HPLM).

41. The method of any one of claims 32-40, wherein the infusion and/or irrigation medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium.

42. The method of any one of claims 32-41, wherein the infusion and/or irrigation medium further comprises one or more antibiotic ingredients.

43. The method of claim 42, wherein the antibiotic ingredient is cefazolin, vancomycin, tobramycin, gentamicin, or amphotericin.

44. The method of any one of claims 32-43, wherein the tissue comprises one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma.

45. The method of any one of claims 32-44, wherein the cells of the tissue contacted by the infusion and/or irrigation medium have greater viability compared to cells of a second tissue contacted by an intravenous-grade saline solution.

46. The method of any one of claims 32-45, wherein the healthcare or veterinarian setting is a sterile environment.

47. The method of any one of claims 32-46, wherein the healthcare setting is an organized provision of medical care to individuals, or to a community.

48. The method of any one of claims 32-46, wherein the veterinarian setting is an organized provision of medical care to animals.

49. The method of any one of claims 32-48, wherein the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

50. A method of providing an intravenous infusion to a subject, comprising intravenously providing a composition comprising tissue culture medium that does not contain serum or growth factors.

51. The method of claim 50, wherein the composition further comprises one or more antibiotic ingredients.

52. A method of irrigating a tissue of a subject, the method comprising exposing the tissue to a liquid medium that does not contain serum or growth factors.

53. The method of claim 52, wherein the liquid medium does not contain peptides.

54. A method of providing an intravenous infusion to a subject, comprising intravenously providing a composition comprising tissue culture medium that does not contain serum or growth factors.

55. The method of claim 54, wherein the tissue culture medium does not contain peptides.

56. A method of irrigating a tissue of a subject, the method comprising exposing the tissue to a liquid medium that does not contain peptides or serum.

57. A method of providing an intravenous infusion to a subject, comprising intravenously providing a composition comprising tissue culture medium that does not contain peptides or serum.

58. A method of storing tissue obtained from a subject, the method comprising exposing the tissue to a tissue culture medium that does not contain serum, or growth factors, and reserving the exposed tissue for re-implantation in the subject.

59. The method of claim 58, wherein the tissue culture medium does not contain peptides.

60. The method of claim 58 or 59, wherein the tissue is stored for less than about 96 hours after obtaining the tissue from the subject.

61. The method of any one of claims 58-60, wherein the subject is a human or a non-human mammal.

62. The method of any one of claims 58-61, wherein the tissue culture medium is Dulbecco's Modified Eagle Medium (DMEM) or alpha-minimum essential medium (αMEM) or similar compositions thereof.

63. The method of any one of claims 58-62, wherein the tissue culture medium is a sterile enriched cell culture medium.

64. The method of any one of claims 58-63, wherein the tissue culture medium is (a) a solid that is reconstituted at the time of use with the addition of a liquid available in the healthcare environment; (b) a solid that is reconstituted at the time of use with the addition of a pre-packaged liquid; or (c) a ready-to-use liquid medium.

65. The method of any one of claims 58-64, wherein the tissue culture medium further comprises one or more antibiotic ingredients.

66. The method of claim 65, wherein the antibiotic ingredient is cefazolin, vancomycin, tobramycin, gentamcin, or amphotericin.

67. The method of any one of claims 58-66, wherein the tissue is a non-whole-organ tissue.

68. The method of any one of claims 58-67, wherein the tissue is a stem cell-containing tissue or a cell fraction.

69. The method of any one of claims 58-68, wherein the stem cell-containing tissue is bone marrow aspirate or bone marrow aspirate concentrate.

70. The method of any one of claims 58-69, wherein the tissue is for use in an autograft in a healthcare or veterinarian setting.

71. The method of claim 70, wherein the tissue comprises one or more of the following: nerve, brain, tendon, ligament, cartilage, muscle, bone, skin, fat, liver, kidney, heart, lung, gastrointestinal tract tissue, reproductive tissue, blood vessels, endothelium, bone marrow and cellular fractions thereof, and plasma.

72. The method of claim 71, wherein the plasma is platelet-rich plasma.

73. The method of any one of claims 58-70, wherein the tissue is an avulsed, amputated, or severed body part, or an intentionally harvested tissue.

74. The method of claim 73, wherein the avulsed or amputated body part is one or more body parts selected from fingers, hands, forearms, arms, toes, feet, legs, ears, scalp, face, lips, penis, tongue and teeth.

75. The method of any one of claims 58-74, wherein the cells of the tissue stored in the tissue culture medium have greater viability compared to cells of a second tissue stored in saline or the University of Wisconsin Cold Storage solution (Belzer UWR Cold Storage Solution).

76. The method of any one of claims 58-75, wherein the cells of the tissue do not significantly proliferate in the tissue culture medium.

77. The method of any one of claims 58-76, wherein the healthcare or veterinarian setting is a sterile environment.

78. The method of any one of claims 58-77, wherein the healthcare setting is an organized provision of medical care to individuals, or to a community.

79. The method of any one of claims 58-77, wherein the veterinarian setting is an organized provision of medical care to animals.

80. The method of any one of claims 58-78, wherein the healthcare or veterinarian setting is an operating room, an outpatient clinical setting, an emergency medical response setting, inpatient hospital setting, a doctor's office, a veterinarian's office, a field office, a procedural suite, a nursing office, a mass sporting event, an ambulance, and in the military field.

81. A method of storing tissue obtained from a subject, comprising exposing the tissue to a composition comprising tissue culture medium that does not contain serum, or growth factors, and reserving the exposed tissue for re-implantation in the subject in a healthcare or veterinarian setting, wherein the tissue is stored for less than about 96 hours after obtaining the tissue from the subject.

82. The method of claim 81, wherein the tissue culture medium does not contain peptides.

83. The method of claim 81 or 82, wherein the composition further comprises (a) a temperature-controlled packaging, or (b) a chemically-activated cool-pack.

84. The method of claim 83, wherein the composition further comprises one or more antibiotic ingredients.

85. The method of claim 84, wherein the antibiotic ingredient is cefazolin, vancomycin, tobramycin, gentamcin, or amphotericin.

86. A composition comprising a tissue culture storage medium, wherein the tissue culture storage medium comprises the following components: water, glucose or fructose, sodium, potassium, chloride, bicarbonate, L-alanine, L-arginine, L-asparagine, L-aspartic acid, L-cysteine, L-glutamic acid, L-glutamine, glycine, L-proline, L-serine, and L-tyrosine; wherein the tissue culture storage medium does not contain serum, or growth factors.

87. The composition of claim 86, wherein the tissue culture storage medium does not contain peptides.

88. The composition of claim 86 or 87, wherein the tissue culture storage medium further comprising a combination of one or more of the following components: lactate, acetate, gluconate, thiamine, riboflavin, niacin, pantothenic acid, pyridoxine, biotin, folates, cobalamin, vitamin A, vitamin D, calcium, vitamin E, vitamin K, vitamin C, L-lysine, L-histidine, L-isoleucine, L-leucine, L-methionine, L-phenylalanine, L-threonine, L-tryptophan, L-valine, glutamine, taurine, glucuronolactone, carnitine, creatine, sodium pyruvate, adenine, adenosine, deoxyadenosine, guanine, guanosine, deoxyguanosine, cytosine, cytidine, deoxycytidine, thymine, thymidine, deoxythymidine, uracil, dietary fatty acids (including saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), lipoic acid, cholesterol, bisphosphonates, and/or inorganic ionic forms of elemental magnesium, calcium, boron, zinc, copper, iron, phosphorous, vanadium, strontium, silica, silicon, selenium, nickel, or manganese; wherein the composition has a near-iso-osmotic balance of 170 to 406 mOsm/L and a pH of 6.0 to 8.8 as a sterile aqueous solution.

89. The composition of any one of claims 86-88, wherein the tissue culture storage medium has a pH of 5.0 to 9.0.

90. The composition of any one of claims 86-89, wherein the tissue culture storage medium comprises the following components: water, glucose and/or fructose (500 to 8000 mg/L), L-valine (0.01-10 mM), L-leucine (0.01-10 mM), L-isoleucine (0.01-10 mM) L-threonine (0.01-10 mM), L-methionine (0.01-10 mM), L-phenylalanine (0.01-10 mM), L-tryptophan (0.005-5 mM), L-lysine (0.1-10 mM), sodium, potassium, chloride, and bicarbonate.

91. The composition of any one of claims 86-89, wherein the tissue culture storage medium comprises the following components: water 96 to 99.5% w/v; glucose and/or fructose 0.05 to 1.0% w/v; L-valine 0.000117 to 0.117% w/v; L-leucine 0.000131 to 0.131% w/v; L-isoleucine 0.000131 to 0.131% w/v; L-threonine 0.000119 to 0.119% w/v; L-methionine 0.000149 to 0.149% w/v; L-phenylalanine 0.000165 to 0.165% w/v; L-tryptophan 0.000102 to 0.102% w/v; L-lysine (0.01-10 mM)=0.000146 to 0.146% w/v; Sodium ion 0.23 to 0.42% w/v; potassium ion 0.0039 to 0.031% w/v; chloride ion 0.21 to 0.53% w/v; bicarbonate ion 0.03 to 0.48% w/v; wherein the composition has a near-iso-osmotic balance of 170 to 406 mOsm/L).

92. The composition of any one of claims 86-91, wherein the composition further comprises (a) a temperature-controlled packaging, or (b) a chemically activated cool-pack.

93. The composition of any one of claims 86-92, wherein the composition further comprises one or more antibiotic ingredients.

94. The composition of claim 93, wherein the antibiotic ingredient is cefazolin, vancomycin, tobramycin, gentamcin, or amphotericin.

95. Use of a tissue culture medium to store tissue obtained from a subject, wherein the tissue is for use in re-implantation in the subject.

96. Use of a composition comprising a sterile tissue culture medium to store tissue obtained from a subject, wherein the tissue culture medium does not contain serum or growth factors, wherein the tissue is stored for less than about 96 hours after obtaining the tissue, wherein the tissue is for use in re-implantation in the subject in a healthcare or veterinarian setting.

97. The use of claim 95 or 96, wherein the sterile tissue culture medium does not contain peptides.

98. A method of preserving cells or tissue obtained from a human subject between the time the cells or tissue are obtained from the subject and re-implantation of the cells or tissue in the subject, the method comprising exposing the removed cells or tissue to a tissue culture storage medium comprising water, glucose or fructose, sodium, potassium, chloride, bicarbonate, L-alanine, L-arginine, L-asparagine, L-aspartic acid, L-cysteine, L-glutamic acid, L-glutamine, glycine, L-proline, L-serine, and L-tyrosine; and wherein the tissue culture storage medium does not contain serum or growth factors.

99. The method of claim 98, wherein the tissue culture storage medium does not contain peptides.

100. The method of claim 98 or 99, wherein the period between obtaining and re-implantation of the cells or tissue is less than about 96 hours.
